(19) United States
(12) Patent Application Publication
(10) Pub. No.: US 2007/0077660 A1
(43) Pub. Date: Apr. 5, 2007

(54) Method for the detection of a cannabinoid, detection kit, and developing solvent

(76) Inventor: Ronald Johannes Glas, Veenendaal (NL)

Correspondence Address: YOUNG & THOMPSON, 745 SOUTH 23RD STREET, 2ND FLOOR, ARLINGTON, VA, 22202, US

(21) Appl. No.: 11/238,974

(22) Filed: Sep. 30, 2005

(51) Int. Cl. G01N 33/00

(52) U.S. Cl. 436/93

(57) ABSTRACT

The invention relates to a method and detection kit for the detection of a cannabinoid in a cannabis sample. The method is based on TLC and the method and kit enables everyone to avoid laborious, time consuming and expensive analytical research and determine the cannabinoid content. Good results are obtained with a developing solvent with at least 25 vol. % chloroform and at least 25 vol. % 1,2-dichloro ethane.

### BACKGROUND

The present invention relates to a method for the detection of a cannabinoid in cannabis, a detection kit for the detection of a cannabinoid in cannabis and to a developing solvent for analyzing cannabis by thin layer chromatography.

### STATE OF THE ART

Marijuana, hashish, sinsemilla, and other psychoactive products obtained from Cannabis sativa are the most widely used illicit drugs in the world. In the United States, smoked marijuana is the primary route of cannabinoid exposure, but in many countries, hashish predominates. Public interest in Cannabis as medicine has been growing, based on the fact that Cannabis has long been considered to have medicinal properties, ranging from treatment of cramps, migraines, convulsions, appetite stimulation and attenuation of nausea and vomiting. Cannabis appear to be useful in treating pain, nausea, AIDS-related weight loss or “wasting”, muscle spasms in multiple sclerosis as well as other problems. Advocates of medical marijuana argue that it is also useful for glaucoma, Parkinson's disease, Huntington's disease, migraines, epilepsy and Alzheimer's disease. Marijuana refers to varieties of Cannabis having a high content of Δ⁹-tetrahydrocannabinol (Δ⁹-THC or THC), which is a psychoactive ingredient of marijuana whereas industrial hemp refers to varieties of the Cannabis plant that have a low content of THC. Cannabinoids share effects with other psychoactive drugs, yet possess a distinct pattern of effects that distinguishes this unique pharmacological drug class.

It would be helpful to those who grow, sell, prescribe, or use, or on the other hand combat the use or import of cannabis, like e.g. pharmacies, hospitals and physicians, consumers, patients, or customs, respectively, if the cannabis can easily be analyzed on e.g. the kind of active species present in the cannabis, the amount of active species and/or the origin of the cannabis. Laboratories may offer the analysis of cannabis, but usually this is relatively expensive, and the client has to wait days to weeks before he receives the expensive analysis report.

In the art, a number of methods are described to analyze cannabinoids. Cannabinoids are the active ingredients in the cannabis, like e.g. cannabichromene (CBC), cannabidol (CBD), cannabidivarol (CBDV), cannabigerol (CBG), cannabinol (CBN), tetrahydrocannabinol (THC), tetrahydrocannabichromene (THCBC), tetrahydracannabidol (THCBD), tetrahydrocannabidivarol (THCBDV), tetrahydrocannabigerol (THCBG), and tetrahydrocannabivarin (THV). An example of an analysis method is described in U.S. Pat. No. 5,252,490, wherein the method comprises analyzing samples of marijuana plant material by gas chromatography/mass spectrometry. Though this may be a routine analysis for specialized laboratories, this method requires GC/MS, which may not be available to home users or pharmacies, etc. U.S. Pat. No. 6,403,126 describes a method for preparing a cannabis extract using a chromatographic column. A number of solvents may be used, including supercritical fluids (e.g. CO₂). Hence, also this document does not provide an easy and cheap method for the detection of a cannabinoid.

Hence, it is an aspect of the invention to provide an alternative method for the detection of a cannabinoid in cannabis which is preferably relatively easy, cheap, and quick. It is a further aspect of the invention to provide a detection kit for the detection of a cannabinoid in cannabis and to provide a developing solvent for analyzing cannabis by thin layer chromatography, that can be used for the detection method of the invention.

### SUMMARY OF THE INVENTION

According to an aspect of the invention, there is provided a method for the detection of a cannabinoid in cannabis comprising:
a) extracting a sample of cannabis with an extraction solvent to provide an extract;
b) providing at least part of the extract to a thin layer chromatography plate;
c) developing a chromatogram by performing thin layer chromatography, thereby using a developing solvent, wherein the developing solvent comprises chloroform and 1,2-dichloro ethane; and
d) determining from the developed chromatogram and reference data the presence and optionally the amount of the cannabinoid in the cannabis.

According to a further aspect of the invention, there is provided a detection kit for the detection of a cannabinoid in cannabis comprising:
a) a container containing at least an extraction solvent for extracting a sample of cannabis;
b) a thin layer chromatography plate; and
c) a container containing at least a developing solvent for developing a thin layer chromatogram, wherein the developing solvent comprises chloroform and 1,2-dichloro ethane.

According to yet a further aspect of the invention, there is provided a developing solvent for analyzing cannabis by thin layer chromatography comprising at least 25 vol. % chloroform and at least 25 vol. % 1,2-dichloro ethane, relative to the total volume of the developing solvent.

According to yet another aspect of the invention, a developing solvent is applied in the method or kit of the invention, wherein the developing solvent comprises one or more halogen substituted hydrocarbons selected from the group consisting of halogen substituted ethane, halogen substituted propane and halogen substituted butane, wherein the hydrocarbon comprises at least two halogen groups, and wherein the developing solvent optionally further comprise chloroform.

### BRIEF DESCRIPTION OF THE DRAWINGS

FIG. 1 schematically depicts an embodiment of a cannabis sample container for weighing a predetermined amount of the cannabis sample;

FIG. 2 schematically depicts an embodiment of an extraction solvent container and a means for providing a predetermined volume of extraction solvent to an extraction container;

FIG. 3 schematically depicts an embodiment of an extraction container, further containing a filtering means, and an embodiment of a spotting container;

FIG. 4 schematically depicts an embodiment of a TLC plate with spots;

FIG. 5 schematically depicts an embodiment of heating the cannabis sample before developing the chromatogram;

FIGS. 6a and 6b schematically depict an embodiment of a developing solvent container and an embodiment of a TLC developing container containing developing solvent and a TLC plate;

FIGS. 7a, 7b and 7c schematically depict embodiments of visualizing the developed spots on the TLC plate and an embodiment of a visualizing agent container;

FIG. 8 shows samples of developed plates;

FIG. 9 shows an example of a reference plate for use as reference for determining the amount of cannabis in a cannabis sample.

### DESCRIPTION OF INVENTION

Cannabis preparations include e.g. loose marijuana plant material, kilo bricks (the classical Mexican-produced material), buds, sinsemilla, Thai sticks, hashish (cannabis resin), hash oil, etc. Δ⁹-tetrahydrocannabinol (THC), the primary psychoactive component, is found in the plant's flowering or fruity tops, leaves, and resin. Cannabis samples can be prepared as known to the person skilled in the art, e.g. by extraction from flower tips, drying and grinding flower tips, etc. Especially THC, one of the active ingredients, is used for its activity. However, for medical purposes specific cannabinoid profiles (fingerprints) may determine the pharmacological effects e.g. glaucoma, relieving spasm/SM (CBD varieties), analgesic (THC, THC varieties, THV varieties), sleep/sedative and anti-vomiting (CBD, CBN, CBND varieties) etc. A cannabis fingerprint (profile) can be used to predict the working behavior of the corresponding cannabis.

The ratio of cannabinoids in cannabis is amongst others dependent on the age of the sample, its geographic origin, the plant strain (its genes) and growing conditions. Cannabinol is essentially a chemical degradation product and its relative abundance increases as samples age. The potency of a preparation is usually described by its THC concentration, e.g. as the % THC per dry weight of material. The actual amount of drug administered will depend upon the weight of the material and the route of drug administration.

Cannabis contains over about 400 different chemical compounds, including 61 cannabinoids. During smoking, more than about 2000 compounds may be produced by pyrolysis. About eighteen different classes of chemicals, including nitrogenous compounds, amino acids, hydrocarbons, sugars, terpenes, and simple and fatty acids contribute to cannabis's known pharmacological and toxicological properties. Other cannabinoids include cannabinol, which is approximately 10% as psychoactive as THC and cannabidiol.

As used herein, the terms “cannabinoid” or “cannabinoids” refer to a family of natural products that usually contain a 1,1′-di-methyl-pyrane ring, a variedly derivatized aromatic ring and a variedly unsaturated cyclohexyl ring and their immediate chemical precursors. In a preferred embodiment, the cannabinoid is selected from the group consisting of cannabichromene (CBC), cannabidol (CBD), cannabidivarol (CBDV), cannabigerol (CBG), cannabinol (CBN), tetrahydrocannabinol (THC), tetrahydrocannabichromene (THCBC), tetrahydrocannabidol (THCBD), tetrahydrocannabidivarol (THCBDV), tetrahydrocannabigerol (THCBG), and tetrahydrocannabivarin (THV), and their corresponding acids. In the invention, the term “cannabinoid” also refers to a number of cannabinoids, i.e. one or more cannabinoid(s), since the method of the invention can also be used to analyze a number of cannabinoids (and/or their corresponding acid(s)) simultaneously with thin layer chromatography (TLC). Hence, the present invention also relates to a method for the detection of cannabinoid(s) in cannabis, a detection kit for the detection of cannabinoid(s) in cannabis and to a developing solvent for analyzing cannabis by thin layer chromatography. Herein the term “fingerprint” especially relates to results of the method of the invention which give information on the type of cannabinoids (including the acids) and/or number of cannabinoids (i.e. qualitative analysis), but may also relate to the concentration of the cannabinoids present in the sample (i.e. quantitative analysis). Hence, fingerprint may refer to both a) information on the type (and number) and b) information on the type (and number) and concentration of cannabinoid(s) present in the cannabis. This information or these informations are a kind of fingerprint of the cannabis sample.

For the method of the invention, a sample can be taken from the cannabis (cannabis preparation) and about 10-500 mg of the cannabis already suffices. At least part of the total content of cannabinoids is extracted from the sample with an extraction solvent to provide an extract. In a preferred embodiment, the extraction solvent comprises chloroform. In an even more preferred embodiment, at least 50 vol. % of the extraction solvent comprises chloroform, more preferably between about 80-100 vol. % of the extraction solvent comprises chloroform, even more preferably between about 95-100 vol. % of the extraction solvent comprises chloroform, relative to the total volume of the extraction solvent. In an embodiment, the extraction solvent consists of chloroform.

The extraction solvent may also comprise, or may in addition to chloroform (if less then 100 vol. % chloroform is used) comprise another organic solvent (or combination of solvents), preferably selected from the group consisting of a low molecular weight alcohol (e.g. a C₂-C₄ alcohol like e.g. ethanol, isopropyl alcohol), a low molecular weight chlorinated hydrocarbon (e.g. methylene chloride, or C₂-C₄ with one or more chlorine side groups like dichloro ethane, e.g. 1,2-dichlore ethane), a petroleum derived hydrocarbon (e.g. petroleum ether 40-60° C., toluene, or trimethyl pentane) or a mixture of two or more of these organic solvents. An advantage of using extraction solvent essentially comprising chloroform is that a high extraction degree is obtained. Cell walls of the cannabis plant seems to be broken and the cannabinoids extracted, whereas conventional solvents like ethanol have not such a high extraction degree (about 1-2% wt. more THC is extracted with chloroform as solvent in comparison to ethanol).

Having extracted at least part of the total amount of cannabinoids present in the cannabis, the extract is applied to a thin layer chromatography plate, and a chromatogram is developed by performing thin layer chromatography, thereby using a developing solvent as mobile phase.

As known to the person skilled in the art, thin-layer chromatography is a very commonly used technique for identifying compounds and determining their purity. In comparison with column chromatography, it only requires small quantities of the compound and is much faster as well. As stationary phase, a special finely ground matrix (e.g. silica gel, alumina, or similar material) is coated on a glass plate, a metal or a plastic film as a thin layer (˜0.25 mm). In addition a binder like gypsum may be mixed into the stationary phase to make it stick better to the slide. In many cases, a luminescent powder is mixed into the stationary phase to simplify the visualization later on (e.g. bright green when exposed to 254 nm UV light). The plate can briefly be spotted at the start line with a capillary filed with the extract. A TLC plate can be developed in a developing container like a beaker or jar. A small amount of solvent (=mobile phase) is placed in the container. The solvent level has to be below the starting line of the TLC, otherwise the spots will dissolve away. The lower edge of the plate is then dipped in a solvent. The solvent (eluent or developing solvent) travels up the matrix by capillarity, moving the components of the samples at various rates because of their different degrees of interaction with the matrix (=stationary phase) and solubility in the developing solvent. Herein, TLC plate may also refer to HPTLC plate (high performance thin layer chromatography).

TLC plates based on silica gel can be used, like e.g. the commercially available Merck silica gel G-60 coated (about 0.2 mm) TLC plates (dimensions 5*10 cm). In another embodiment, HPTLC plates, e.g. of Machery Nagel, are used. In an embodiment, the (HP) TLC plates are based on thin layers selected from the group consisting of silica gel G-60, silica gel G-150 and modifications thereof. In another embodiment, thin layers of cellulose are used. In yet a further embodiment, the TLC plate comprises cellulose (i.e. paper without a support material). Preferably, the plate length is chosen such that the developing solvent can be allowed to travel at least 5 cm, more preferably at least between about 5-10 cm from the initial spot at the TLC plate. As known to the person skilled in the art, usually the height of the liquid above the bottom of a container wherein the plate is placed is about 0.1-1 cm. The initial spot is usually positioned at the TLC plate such that when the plate is arranged in a container with developing solvent, the spot is about 0.2-1 cm above the surface of the developing solvent.

In an embodiment of the invention, the developing solvent comprises chloroform and 1,2-dichloro ethane. Developing solvents of the art may e.g. comprise ethanol. However, these developing solvents do not have the high resolution of the developing solvent of the invention and do not provide a good chromatogram of the cannabinoid(s) in a relatively short time. Hence, the developing solvent of the invention used in the method of the invention may enable a relatively easy, cheap and quick (about 10-30 minutes) determination of the cannabinoids present in the cannabis. For example, when using the “slightly” different molecule 1,1-dichloro ethane, substantially worse results are obtained since the developing time is larger and the Rf values of the cannabinoids appear to be relatively close to each other (which is especially a problem for species with high cannabinoid contents) and the acids have even overlapping Rf values, thereby substantially complicating analysis of the cannabis sample.

In a specific embodiment, the developing solvent (mobile phase) comprises at least 25 vol. % chloroform and at least 25 vol. % 1,2-dichloro ethane, relative to the total volume of the developing solvent, more preferably at least 40 vol. % chloroform and at least 40 vol. % 1,2-dichloro ethane. The developing solvent may in addition to (the at least 25 or 40 vol. %, respectively) chloroform and (the at least 25 or 40 vol. %, respectively) 1,2-dichloro ethane comprise organic solvents known to the person skilled in the art, preferably selected from the group consisting of a low molecular weight alcohol (e.g. a C₂-C₄ alcohol like e.g. ethanol, isopropyl alcohol); other low molecular weight chlorinated hydrocarbon (e.g. methylene chloride, or C₂-C₄ with one or more chlorine side groups like 1,1-dichloro ethane); a petroleum derived hydrocarbon (or a mixture thereof, e.g. containing one or more of petroleum ether(s), in an embodiment the petroleum derived hydrocarbon (or mixture of hydrocarbons) having a boiling range of about 40-120° C., in a specific variant 40-60° C.; toluene; and trimethyl pentane); a low molecular weight at room temperature liquid hydrocarbon, preferably C₅-C₈, like heptane; benzene; mono, di or tri methyl benzene, like e.g. xylene; dioxane solvents like e.g. 1,3- or 1,4-dioxane; isomers of these compounds; or a mixture of two or more of these organic solvents, but may also contain other solvents known to the person skilled in the art.

More preferably, the developing solvent contains 25-75 vol. % chloroform and 75-25 vol. % 1,2-dichloro ethane, relative to the total volume of the developing solvent, even more preferably, the developing solvent contains 30-70 vol. % chloroform and 70-30 vol. % 1,2-dichloro ethane, yet even more preferably, the developing solvent contains 40-60 vol. % chloroform and 60-40 vol. % 1,2-dichloro ethane, relative to the total volume of the developing solvent, like 50%/50%. In these embodiments, the developing solvents essentially consists of the two components chloroform and 1,2-dichloro ethane.

Having developed the chromatogram (i.e. when the developing solvent has been allowed to travel up the plate at least about 5 cm from the initial spot), the developed spots will usually be visualized by e.g.:
1. Sulfuric acid/heat: leaves charred blots behind;
2. Ceric stain (ceric ammonium molybdate (CAM)): leaves a dark blue blot behind for polar compounds;
3. Iodine: iodine absorbs onto the spots, giving coloration;
4. Dye: an organic dye can be used which leaves to a colorization of the spots;
5. UV light: e.g. 254 nm, with luminescent plate the background is e.g. green and the spots on the developed TLC plate are dark. Further, the cannabinoids may luminescence or the dye/colorant sprayed on the TLC-plate may provide luminescent spots (at the place of the spots on the developed TLC plate).

In a preferred embodiment for determining the presence of the cannabinoid or the amount of the cannabinoid or both a visualizing agent (dye) is sprayed over the developed chromatogram. In an embodiment of the invention, o-dianiside, tetrazotized zinc chloride double salt (diazo fast blue B salt, C₁₄H₁₂N₄O₂Cl₂.ZnCl₂, CAS Number 14263-94-6 (Fluka 44660); e.g. in aqueous solution 0.3% (w/v)) is used as dye. Especially this dye has very low detection limits for cannabinoids, e.g. down to 0.01 μg and distinct colorations of the cannabinoids on the TLC plate. Also other dyes or colorants which may give a coloration of one or more of the above mentioned cannabinoids and/or their corresponding acids, can be used. For example, also dapsone, aniline, p-chloroanilin, p-toluidin, sulphadiazin, o-aminobenzoic acid, HMBT can be used, but also cupric salts, ninhydrin, molybdenum blue reagent, vanillin, potassium permanganate and fluorescent dyes, including but not limited to primulin may be used. The person skilled in the art may test and find appropriate dyes/stains. Hence, the phrase “determining from the developed chromatogram the presence of the cannabinoid (one or more cannabinoid(s)) may include a visualizing step. In this step, the separated components on the developed chromatogram are visualized by means of e.g. a dye, stain, etc., as mentioned above, or by means of a UV lamp (see below).

In another preferred embodiment, visualization is performed by irradiating the plate with UV light. Preferably, a luminescent TLC plate is used, i.e. a TLC plate comprising a luminescent material, as known to the person skilled in the art. Hence, in a preferred embodiment for determining the presence of the cannabinoid or the amount of the cannabinoid or both a UV source is used for irradiating and visualizing a spot of the cannabinoid on the developed chromatogram.

The components, visible as (separated) spots, are identified by comparing the distances they have traveled with those of known reference materials. To this end, information in or on a medium may be provided with respect to the Rf-values (retardation factor) or RRf values (relative retardation factor) or with respect to both Rf and RRf of the cannabinoids and/or the corresponding acids under the conditions the method for the detection is to be performed. Alternatively, one or more reference TLC plate(s) or copies thereof (as information on mediums), can be provided, which shows the position of the spots of at least one of the cannabinoids or a number of cannabinoids and/or the corresponding acids, and which has been made under similar conditions (i.e. same developing solvent; same kind of TLC plate, same end height of developing solvent on TLC plate and same temperature, e.g. room temperature). Hence, the kit may further comprise a medium comprising reference data, wherein the reference data comprise information on the retardation factor of the cannabinoid(s).

The method of the invention can be used to make a fingerprint of the cannabinoid, thereby providing information of the cannabinoids present in the cannabis. However, the method of the invention can also be used to determine the amount of the cannabinoid in the cannabis since the area of the spot of a cannabinoid on a developed TLC plate corresponds to the amount of cannabinoid present in the sample (i.e. in the cannabis). To this end, the kit may comprise a medium comprising reference data, wherein the reference data comprise information on i) the relation of spot area of the cannabinoid on the developed chromatogram and amount of the cannabinoid(s) in the cannabis, or ii) the relation of spot intensity on the developed chromatogram and amount of the cannabinoid(s) in the cannabis or iii) the relation of spot area and spot intensity of the cannabinoid on the developed chromatogram and amount of the cannabinoid(s) in the cannabis. For example information in or on a medium may be provided with respect to the relation of the spot size and/or intensity of the color of the spot and concentration of the cannabinoids and/or the corresponding acids under the conditions the method for the detection is to be performed. Alternatively, one or more reference TLC plate(s) or a copies thereof (as information on mediums), can be provided, which shows of at least one of the cannabinoids or a number of cannabinoids, the spots with their specific sizes (and/or intensities), and which has been made under similar conditions (i.e. same developing solvent; same kind of TLC plate, same end height of developing solvent on TLC plate and same temperature, e.g. room temperature) and information on the corresponding cannabinoid(s) content(s). Only using the spot size already gives a rather reliable result on the concentration of the cannabinoid(s) in the sample. However using spot size and spot intensity (intensity of the color) give even better results.

Hence, according to the invention, the kit may further comprise a medium comprising reference data, wherein the reference data comprise one or more of a) information on the retardation factor (Rf and/or RRf) of the cannabinoid, b) the relation of spot area of the cannabinoid on the developed chromatogram and amount of the cannabinoid in the cannabis, c) the relation of spot intensity on the developed chromatogram and amount of the cannabinoid in the cannabis and d) the relation of spot area and spot intensity of the cannabinoid on the developed chromatogram and amount of the cannabinoid in the cannabis. Preferably, the reference data comprise a) information on the retardation factor (Rf and/or RRf) of the cannabinoid (fingerprint). For a quantitative analysis, preferably, the reference data comprise a) information on the retardation factor of the cannabinoid and b) the relation of spot area of the cannabinoid on the developed chromatogram and amount of the cannabinoid in the cannabis; more preferably the reference data comprise a) information on the retardation factor (Rf and/or RRf) of the cannabinoid, b) the relation of spot area of the cannabinoid on the developed chromatogram and amount of the cannabinoid in the cannabis, and c) the relation of spot intensity on the developed chromatogram and amount of the cannabinoid in the cannabis. Hence, in a specific embodiment, the reference data comprise a) information on the retardation factor of the cannabinoid and d) the relation of spot area and spot intensity of the cannabinoid on the developed chromatogram and amount of the cannabinoid in the cannabis.

In yet a further embodiment, determination may be done by scanning the developed TLC plate with a scanner and using a computer program product comprising computer executable instructions, e.g. on the medium (like a disk, memory stick, etc.) which, when loaded on a computer, derives in combination with reference data (which may also be present on the medium) from the spot positions and/or spot areas the kind of cannabinoid(s) present in the cannabis and/or the amount of the respective cannabinoid(s) in the cannabis. To this end, in connection or integrated to the program, there may be a data input function for data concerning one or more selected of the group consisting of starting position on the TLC plate of the developing solvent surface, starting position on the TLC plate of respective spots, end position on the TLC plate of developing solvent and amount (predetermined amount) of cannabis used to provide an extract, such that in combination with the reference data (see above) the type of cannabinoid(s) present and/or the amount of these cannabinoid(s) can be determined.

Hence, according to the invention, the method comprises the step of determining from the developed chromatogram and reference data the presence and optionally the amount of the cannabinoid in the cannabis. The determination step may include a visualization step. The determination step may further include comparing the developed spots on the developed TLC plate with reference data and concluding which cannabinoid or cannabinoids appear to be present in the cannabis sample (i.e. detection of one or more cannabinoids present in the cannabis). Further, from the spot areas and/or spot intensities (intensity of the color) of the separated components on the developed TLC plate and reference data, the amount of the respective cannabinoid(s) can be estimated in the sample (i.e. the cannabis). By e.g. digitally counting pixels of the spots with means and software known to the person skilled in the art estimations of the concentration(s) can be made with an error below 5%.

In yet a further embodiment, the kit may comprise a medium or mediums comprising both reference data on the retardation factor of the cannabinoid and information on the relation of spot area of the cannabinoid on the developed chromatogram and amount of the cannabinoid in the cannabis.

In a specific embodiment, the invention is directed to a method for the detection of a cannabinoid in cannabis comprising:
weighing a part of the cannabis such that a sample is provided with a predetermined weight; and optionally heating the sample;
mixing extraction solvent and the sample;
extracting at least part of the total amount of the cannabinoids in the cannabis sample with the extraction solvent;
optionally filtering the extract;
sampling the optionally filtered extraction liquid with extract to provide a sample to be spotted on the TLC plate;
spotting on the TLC plate an amount of the extraction liquid with the extracted cannabinoid(s); and optionally heating one or more spots on the TLC plate;
developing the TLC plate;
visualizing the separated component(s) on the developed TLC plate;
determination from the developed chromatogram and the reference date the presence, the amount or both the presence and the amount of the cannabinoid(s) in the cannabis sample.

By only an additional heating step, the same method as used for the determination of the cannabinoid(s) present in the cannabis sample, as described above (and below), can be also be used to determine the quantity of these cannabinoid(s). Only one of the optional heating steps may be applied for determining the amount of cannabinoid(s) in the sample.

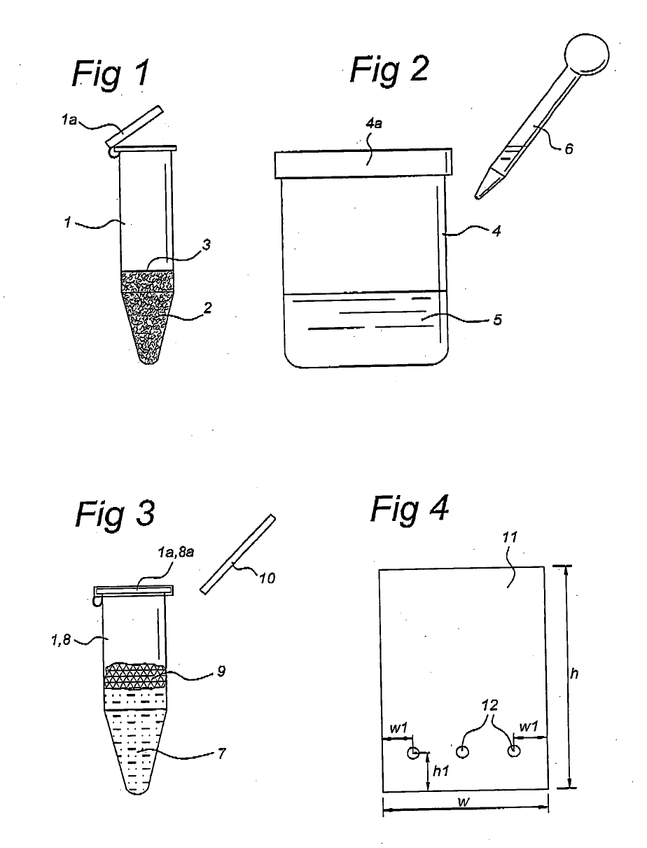

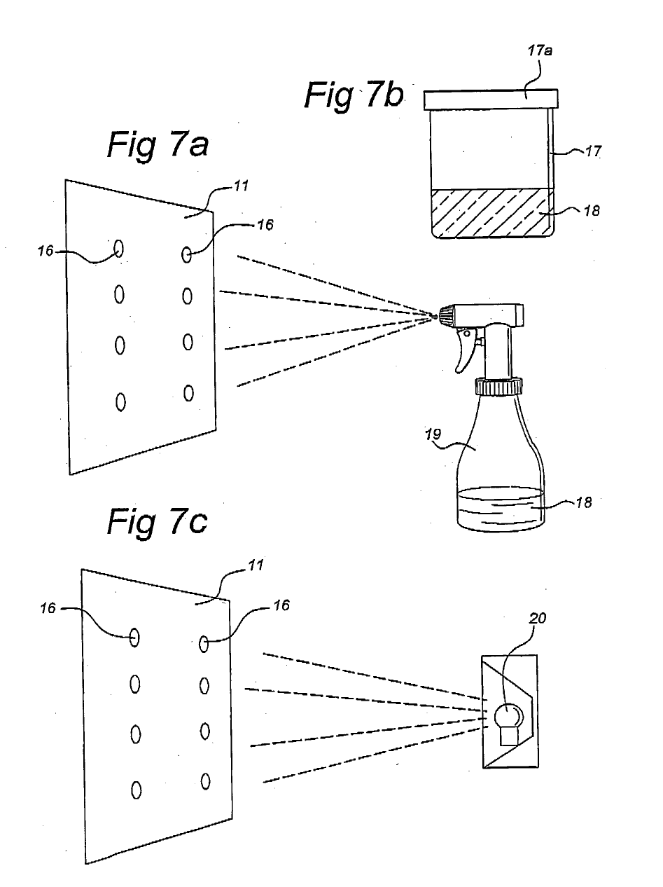

Referring to FIGS. 1-9, this embodiment will be described in more detail below. For weighing a part of the cannabis such that a sample 2 is provided with a predetermined weight a weighing means may be used, like e.g. a balance, but the cannabis (e.g. hashish, marihuana) may also be added to a container (cannabis sample container 1), e.g. an Eppendorf reaction tube (with optional cover 1a), designed such that a predetermined amount can be added to the container. For example, for using an about 100 mg (predetermined amount) cannabis sample 2, the container 1 has a mark 3 which will be reached upon filling the container 1 with the sample 2, such that it is clear to the person performing the analysis or determination when this predetermined amount in the container 1 is reached. In an embodiment, the predetermined amount to be used as cannabis sample 2 is between about 10-500 mg, preferably between about 20 and 250 mg, more preferably between about 50 and 250 mg.

The cannabis sample container 1 may be chosen such that it can also be used as extraction container 8 (see below).

Then, the method is continued with:
mixing extraction solvent and the sample.

From an extraction solvent container 4 (with optional cover 4a), with another container 6, a predetermined volume of the extraction solvent 5 can be added to the sample 2 in an extraction container 8. This container 6 may e.g. be a 1 ml (predetermined volume) pipette such that 1 ml extraction solvent 5 can be added to the to the container 1 (i.e. extraction container 8 in this embodiment) mentioned in step 1 (Eppendorf reaction tube). Preferably, the ratio cannabis sample 2 to solvent 5 is about 10-500:1 (mg cannabis to ml extraction solvent), more preferably between about 20-250:1. Assuming that the predetermined amount to be used as cannabis sample 2 is between about 10-500 mg, the volume of extraction solvent 5 to be added is preferably between about 0.04 ml (20 mg; 500:1) and 50 ml (500 mg; 10:1). For example, 1 ml extraction solvent may be used for 100 mg sample.

Of course, the cannabis sample 2 may also be added to the predetermined volume of the extraction solvent 5, when a suitable container for the predetermined volume of the extraction solvent is used (for example a container 8, which may be different from container 1).

The next step is:
extracting at least part of the total amount of the cannabinoids in the cannabis sample with the extraction solvent.

The extraction solvent S and sample 2 have to be mixed. This may e.g. be performed by closing the extraction container 8 with the extraction solvent 5 and cannabis sample 2 with closing means 1a/8a (depending whether or not the same container is used) and shaking the container 8 (e.g. by hand) for a few minutes, e.g. 1-10 minutes, preferably 2-5 minutes. During shaking, cover 8a (1a) may be closed. If larger amounts than about 100 mg and/or about 1 ml are used, one may shake (or stir) longer, as will be understood by the person skilled in the art. By mixing, e.g. by shaking, the extraction of at least part of the cannabinoids take place, thereby providing an extraction solvent with extract 7. As will be clear to the person skilled in the art, by choosing suitable containers, like e.g. the Eppendorf reaction tube 1, the weighing means for the cannabis sample can be a container 1 for weighing the sample but this container can also be used as extraction container 8.

Then follows the optional step of:
filtering the extract.

This optional step may be applied for filtering cannabis material that does not solve. Filtration may be performed by ways known to the person skilled in the art and is e.g. performed for preventing clogging of a capillary 10 (see below). In a preferred embodiment of the invention, filtration is performed by arranging e.g. cotton 9 (like absorbent cotton/cotton wool or glass wool, etc.) in the container 8 containing the extraction solvent with extract 7 in fluid contact with the liquid surface of the extraction solvent with extract 7. This may be easily done in a small container 8, like a small Eppendorf reaction tube with a volume of e.g. about 1-5 ml. The cotton 9 may be placed “on top” of the greenish (due to chlorophyll) extraction solvent 7 and the cotton 9 gets saturated with the extraction liquid (=filtration).

In an other embodiment, filtration paper is used, and a filtrate is formed in a container.

The method of the embodiment is continued by:
sampling the extraction liquid with extract to provide a sample to be spotted on the TLC plate

A predetermined amount of the extraction liquid with extract 7 (optionally filtered) is to be spotted on the TLC plate 11. To this end, with a next container (“spotting container” 10), part of the (filtered) extraction liquid with extract 7 is sampled. This container 10 may e.g. be a hypodermic syringe, a micro pipette, a capillary tube, designed to sample a predetermined volume of the extraction liquid with extract 7. For example, a capillary tube can be pressed slightly into the saturated cotton. The capillary tube will take up the predetermined amount of extraction liquid, preferably about 0.2-10 μl, more preferably about 0.5-5 μl, like 2 μl, by capillary action. In an embodiment, this is an “end-to-end capillary” which is filled completely, from end to end. When no quantitative analysis is desired and only a finger print is desired, one may also use other means to provide the extraction liquid with extract 7 to the TLC plate 11, like e.g. using the tip of a glass stick as “container” and providing a droplet to the TLC plate 11.

The next step includes the actual action of:
spotting on the TLC plate an amount of the extraction liquid with the extracted cannabinoid(s).

With container 10 described in the previous step, e.g. a capillary, the TLC plate 11 is spotted with the liquid contained in the container 10. Referring to the step above, 2 μl of sample within the capillary tube with 2 μl of extraction liquid is spotted on the TLC plate 11, preferably about a 0.5-1.5 cm (h1) from a bottom of the TLC plate and preferably about ¼ cm (w1) or more from the side edges, more preferably about 1-1.5 cm and about ½ cm or more, respectively. Preferably, for a quantitative analysis a predetermined volume, preferably 0.2-10 μl, is spotted on the TLC plate, such that the developed spot size can be related to the cannabinoid content of the sample. Spots are indicated with reference number 12.

Further, the embodiment is continued with:
developing the TLC plate.

A developing chamber 15 or (“developing container”) is provided, preferably having a smallest width w2 (or diameter w2) substantially the same as the width w of the TLC plate 11 (see FIGS. 4, 6a and 6b), e.g. about 0.5-2 cm broader than width w of the TLC plate 11, and a height h3, i.e. dimensions selected such that plate 11 with height h can stand substantially up right, i.e. receding with angle a between about 0 and 45° from a normal perpendicular to the surface of the developing solvent 14 (when added to the developing chamber or container 15). The person skilled in the art however knows how to perform TLC.

Preferably before placing the TLC plate 11 in the developing chamber or container 15, developing solvent 14 (e.g. contained in developing solvent container 13 with optional cover 13a) is added, such that the bottom of TLC plate 11 (when placed) will be dipped in the fluid for about 1-10 mm, preferably about 2-5 mm. The developing solvent surface is preferably at least 1-5 mm below (h2) the bottom of the spot 12 or spots 12 with extraction liquid on TLC plate 11 (see above); preferably distance h2 is between about 1 and 10 mm, more preferably about 5-10 mm. The developing solvent 14 may be added with a pipette or another type of containing means like a syringe, etc., or by simply pouring the developing solvent 14 from the container 13 containing the developing solvent 14 into the developing chamber or container 15 (preferably before placing TLC plate 11 in container 15), as known to the person skilled in the art. Note that spot(s) 12 in FIG. 6a is (are) indicated with an elevation for the sake of understanding (side view of plate 11).

Preferably, the developing chamber or container 15 is closed with a cover 15a when both the developing solvent 14 and the TLC plate 11 are provided into the chamber or container 15. Then the chromatogram is allowed to develop for about at least about 5-30 minutes, preferably at least about 20-25 minutes, preferably the chromatogram is allowed to develop for the time that is enough for a rise of the front of the liquid on the TLC plate 11 for at least 5 cm, more preferably at least between about 5-10 cm, relative to the initial spot(s) 12 at the TLC plate 11. Development is finished by removing plate 11 from the developing solvent 14, at least when the solvent front has reached the top of plate 11. As will be known to the person skilled in the art, also 2D chromatography may be applied. However, the method according to the invention already provides a good and fast analysis with 1D chromatography. In an embodiment, the developing time is selected such that a person skilled in the art can do a qualitative analysis. In yet a further embodiment, the developing time is selected such that a person skilled in the art can do a quantitative analysis. The developing time used for determining the amount of cannabis will usually be correlated with the reference data, i.e. the developing time, TLC plate 11 (i.e. material and material layer thickness), temperature and developing solvent 14 are substantially the same or equivalent to those used for obtaining the reference date, as will be clear to the person skilled in the art. Hence, there may be different reference data for different developing solvents 14 and TLC plates 11 (and combinations).

Thereafter, TLC plate 11 is removed from the developing chamber 15 and allowed to dry at ambient conditions, although the TLC plate 11 may also gently be heated, e.g. using a blow-drier or hair-drier (preferably <50 ° C. for a few minutes).

Since the developed spots, i.e. the spots that are formed by each cannabinoid on the plate due to the migration on the TLC plate 11 with the developing solvent 14, may not be visible with the human eye, in a further embodiment the method of the invention may also include:
preparing a visualizing agent or providing a visualization means.

The visualizing agent 18 can e.g. be prepared during development. However, the visualizing agent 18 may also be on stock, may be present as ready liquid in the kit, etc. Further, this step may not be necessary in case a TLC plate 11 is used with a luminescent agent, as known in the art, and e.g. a UV source 20 (visualization means, e.g. with 254 nm radiation) is used to determine the spot positions 16 (and/or areas) on the developed plate.

In a further embodiment, an amount of the visualizing agent 18 (dye, colorizing agent or stain) from a container or vial 17 (with optional cover 17a), may be introduced into a nebulizer or sprayer 19, if desired filled with a predetermined amount of a diluent, e.g. water. When a diluent is used, mixing of the diluent and the visualizing agent 18 is preferred.

After developing the TLC plate, the spots 16 on the developed chromatogram have to be visualized. Hence, the method is continued by:
visualizing the separated component(s) on the developed TLC plate.

Assuming the use of a visualizing agent 8 as described above, this agent may be sprayed on the plate 11 with sprayer 19. The bright colored cannabinoid spots 16 appear (nearly) instantaneously. Alternatively, as described above, a UV source 20 as visualizing means can be used.

Having visualized the spots 16 on the TLC plate 11, the method is continued with:
determination from the developed chromatogram and the reference date the presence, the amount or both the presence and the amount of the cannabinoid(s) in the cannabis sample.

For determining the amount of THC, and in a similar way of the other cannabinoid(s), in the cannabis, the invention provides an embodiment wherein the cannabinoids are heated before developing the plate. In general, the specific embodiment as described above may be followed, but with a few modifications. This method may also be used for other cannabinoid(s) like e.g. CBD.

Further, for a quantitative analysis, fixed amounts of cannabis sample, solvents etc. have to be used, in order to compare with the reference data, i.e. same extraction solvent and developing solvent as reference sample, fixed amounts of sample, fixed amount of extraction solvent in order to relate with the reference data, fixed amount of sample on TLC plate (also in order to relate with the reference data). As will be clear to the person skilled in the art, for qualitative analysis (fingerprint), mainly the same choice of developing solvent (and (HP) TLC plate material) is relevant in order to compare with the reference data (Rf).

Before measuring the THC concentration of the sample, the natural occurring cannabinoids in the sample must be heated first in order to remove the acid groups. i.e. “—COOH” attached to the molecule (e.g. THCA→THC). By this action, all the (reddish) cannabinoid acids which will usually be found low in the plate will not be found. In comparison to a non-heated sample, the THC spot of a heated sample will increase in width and intensity (see also FIG. 9).

Therefore, all cannabinoid products must be heated, cooked, boiled or baked before developing the plate 11 in order to transform the pharmacologically and psychotropic inactive cannabinoid acids into the biologically active derivatives. The latter ones will appear always above the acids.

According to the method of the invention, there are a number of alternatives to heat the cannabinoid sample. In a specific embodiment, the method of the invention further includes:
weighing a part of the cannabis such that a sample is provided with a predetermined weight, followed by a heating of the sample.

In an embodiment, the sample may be placed on a little piece of aluminum foil or on a small dish or in a crucible, etc. Then the sample can be heated in e.g. an oven for about 2-8 minutes, preferably about 3-6 minutes at about 80-200° C., preferably about 120-180° C. By trial and error (i.e. varying heating temperature, heating time; and developing the TLC plate 11 and checking the result), the appropriate conditions can be found. If e.g. different reddish spots appears between the CBN spot and the natural smear of acids on the fingerprint (the not heated version) the temperature was too high and/or the heating period too long. Heating for about 4 minutes at about 150° C. provided good results.

Alternatively, in a specific embodiment, the method of the invention further includes:
spotting on the TLC plate an amount of the extraction liquid with the extracted cannabinoid(s), followed by a heating of the plate.

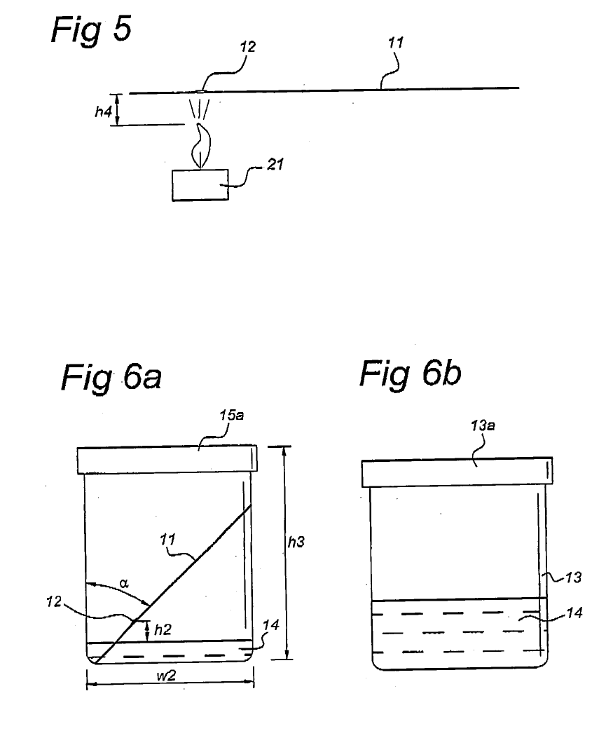

Referring to FIG. 5, after providing one or more spots 12 to the plate 11, as described above, TLC plate 11 can be heated. The same heating conditions as described above can be used, e.g. 4 minutes in an oven at about 150° C. Alternatively, the TLC plate 11 may be carefully heated with a candle or lighter 21. To this end, the plate 11 may be seized between thumb and index-finger or with a tweezers, etc, preferably such that only the edges of the plate 11 are touched. The spots 12 are carefully heated, about 2.5-4 cm (h4) over the flame of candle or lighter 21 for about 1-3 minutes, at both sides of the plate 11 (silica side/glass side). Also for this method applies that some trial and error may be necessary to find the right heating conditions. Good results were obtained with a distance h4 of about 3 cm and a heating time of 1½ minutes. Heating is usually performed either directly after weighing the cannabis sample 2 or after spotting the extraction liquid with the extracted cannabinoid(s) 7 on the TLC plate 11. Hence, for determining the cannabinoid content in the cannabis according to the method of the invention, until development, only one of the optional heating steps as mentioned above is chosen.

One may also choose to provide e.g. two spots 12 on the TLC plate and heat only one of them. When developing, the unheated spot 12 will provide a chromatogram that can be used as fingerprint for qualitative analysis, and the heated spot 12 will provide a chromatogram that can be used for quantitative (and qualitative) analysis.

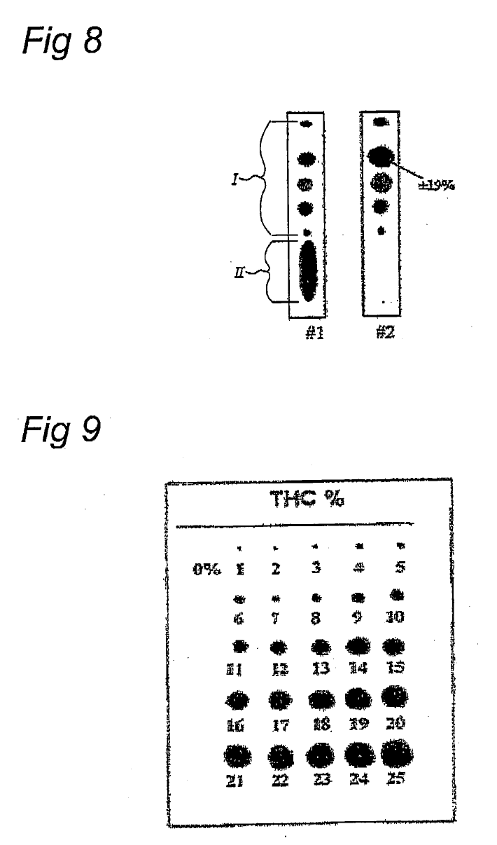

FIG. 8 displays two sample plates. In this embodiment, plate 1 has two regions, wherein the region indicated with I reflects the spots of CBC, THC, CBD, CBN, respectively, and region II reflect the acids. In this figure (with this graphical quality) the acids are smeared out, but depending upon the type of cannabis and the conditions, one or more acids may appear to be separated. Plate 2 reflects the situation that before developing, the sample or spot was heated, such that substantially all the acids are converted. This leads to larger spots in the cannabinoid region I. FIG. 9 provides an embodiment of a reference plate, in this case for THC, but reference data for other cannabinoids can be provided as well.

After optional heating, development and visualization, spots 16 are visible and the track they have traveled on the plate 11 while developing can be determined, Rf/RRf values (see below) can be determined, as known to the person skilled in the art, and can be compared with reference data on these values, or the TLC plate 11 can be compared with a reference plate (as reference data, see e.g. FIGS. 8 and 9), prepared under similar conditions with known cannabinoid(s) (and/or their corresponding acid(s)).

Below, example reference values for Rf and relative Rf values (RRf, with THC as internal reference) are given for a number of cannabinoids and corresponding acids, with chloroform/1,2-dichloro ethane (60 vol. %/40 vol. % at RT) as developing solvent 14 and the color of the spots with o-dianiside as dye 18 and a silicagel G 60 TLC plate:

| | | | | Color of |
|---|---|---|---|---|
| | | | | spot after |
| Cannabinoid | | Rf value | RRf value | spraying |
| Cannabichromene | CBC | 0.92 | 1.43 | Purple; |
| | | | | after 1 hr: |
| | | | | Orange- |
| | | | | Brown |
| Cannabigerol | CBG | 0.70 | 1.10 | Orange |
| Tetrahydrocannabinol | THC | 0.64 | 1 | wine-Red |
| Tetrahydrocannabivarin | THV | 0.56 | 0.87 | wine-Red |
| Cannabidiol | CBD | 0.49 | 0.76 | Yellow |
| Cannabinol | CBN | 0.45 | 0.68 | Purple |
| CBC-acid | CBCA | 0.19 | 0.30 | Purple- |
| | | | | reddish |
| THC-acid | THCA | 0.23-0.05 | 0.36-0.08 | Reddish |
| | | | | smear |
| CBG-acid | CBGA | 0.16 | 0.25 | Orange- |
| | | | | reddish |
| CBD-acid | CBDA | 0.11 | 0.17 | Yellow- |
| | | | | reddish |
| CBN-acid | CBNA | 0.09 | 0.10 | Purple- |
| | | | | reddish |

RRf-values are unique for the used developing solvent, and are independent of e.g. layer thickness of the (HP)TCL plate material (on the (HP) TLC plate support material, as known to the person skilled in the art). Especially when the developing solvent according to the invention is used, the good separation is provided.

According to another aspect of the invention, there is provided a detection kit for the detection of a cannabinoid in cannabis comprising:
a) a container containing at least an extraction solvent for extracting a sample of cannabis;
b) a thin layer chromatography plate;
c) a container containing at least a developing solvent for developing a thin layer chromatogram, wherein the developing solvent comprises chloroform and 1,2-dichloro ethane; and
d) optionally one or more selected of:
   i) one or more container(s) (with cover(s) or another closing means) containing a visualizing agent for the cannabinoid on the developed chromatogram, like a bag or a flask;
   ii) one or more sprayers; and
   iii) a UV source.

Herein, a thin layer chromatography plate may also comprise a number of TLC plates. For example, the kit may contain 10-100 TLC plates.

In a specific embodiment, there is provided a detection kit or box, preferably a cardboard or hard plastic box, containing:
one or more extraction solvent container(s) (with cover(s) or another closing means) containing the extraction solvent for extracting a sample of cannabis;
one or more TLC plate(s);
one or more developing solvent container(s) (with cover(s) or another closing means) containing the developing solvent for developing a thin layer chromatogram, wherein the developing solvent comprises chloroform and 1,2-dichloro ethane;
one or more selected of:
   one or more container(s) (with cover(s) or another closing means), designed to contain a predetermined volume of extraction liquid, e.g. a pipette;
   one or more extract sampling means or spotting container(s) like syringe(s), pipette(s), or capillary tube(s), designed to contain a predetermined volume of the extraction liquid with extract;
   one or more TLC developing container(s) (with cover(s) or another closing means);
   one or more filter means, like e.g. cotton or filter paper(s);
   one or more heating means, preferably selected from the group consisting of a lighter, a candle, a heater and a small furnace;
one or more selected of:
   one or more container(s) (with cover(s) or another closing means) containing a visualizing agent for the cannabinoid on the developed chromatogram, like a bag or a flask;
   one or more sprayers;
   a UV source; and
one or more medium(s) comprising reference data, wherein the reference data comprise information on the retardation factor (Rf and/or RRf) of the cannabinoid(s), or information on the relation of spot area and/or spot intensity of the cannabinoid(s) on the developed chromatogram and amount of the cannabinoid(s) in the cannabis, or both these in formations.

The mediums may be one or more selected from the group consisting of a Rf data sheet, a reference TLC sheet or copy thereof and reference data on a data carrier for loading on a computer, which, when loaded upon the computer provides information on Rf data (or RRf data or both) and/or a reference TLC sheet. Further, the data carrier may comprise a program, which when loaded on a computer, can compare a scanned developed TLC with the reference data and provide output information on the cannabinoid(s) present in the sample or the amount of the cannabinoid(s) present in the sample or both this qualitative and quantitative information (see further also above).
According to the invention, there may also be provided a number of kits, e.g. a first kit comprising:
a container (one or more containers) containing at least an extraction solvent for extracting a sample of cannabis;
a thin layer chromatography plate (one or more plates);
a container (one or more containers) containing at least a developing solvent for developing a thin layer chromatogram, wherein the developing solvent comprises chloroform and 1,2-dichloro ethane, and
one or more selected of:
   one or more container(s) (with cover(s) or another closing means), designed to contain a predetermined volume of extraction liquid, e.g. a pipette;
   one or more extract sampling means or spotting container(s) like syringe(s), pipette(s), or capillary tube(s), designed to contain a predetermined volume of the extraction liquid with extract;
   one or more TLC developing container(s) (with cover(s) or another closing means);
   one or more filter means, like e.g. cotton or filter paper(s);
   one or more heating means, preferably selected from the group consisting of a lighter, a candle, a heater and a small furnace;
one or more selected of:
   one or more container(s) (with cover(s) or another closing means) containing a visualizing agent for the cannabinoid on the developed chromatogram, like a bag or a flask;
   one or more sprayers;
   a UV source; and
one or more medium(s) comprising reference data, wherein the reference data comprise information on the retardation factor of the cannabinoid, or information on the relation of spot area and/or spot intensity of the cannabinoid on the developed chromatogram and amount of the cannabinoid in the cannabis, or both these in formations.

The user of the kit may e.g. order later supplemental or replenishment kits, e.g. a kit comprising a container (one or more containers) containing at least an extraction solvent for extracting a sample of cannabis, a thin layer chromatography plate (one or more plates) and a container (one or more containers) containing at least a developing solvent for developing a thin layer chromatogram, wherein the developing solvent comprises chloroform and 1,2-dichloro ethane, and use elements like the TLC developing container, the UV source, etc. from the first kit.

In a specific embodiment, the developing solvent is also used as extraction solvent. Hence, the container containing at least an extraction solvent for extracting a sample of cannabis comprises the container containing at least a developing solvent for developing a thin layer chrornatogram. This means that the kit of the invention may contain one single container (or more containers) containing the developing solvent as used in the method of the invention. This developing solvent is then also used as extraction solvent.

The cannabinoid (phytochemistry) detection kit and method according to the invention provide the possibility to do research on the cannabinoid fingerprints of cannabis species. By using this kit medicinal marihuana users, growers of hemp and marihuana, recreational users, physicians, customs, forensic experts etc. e.g. get the possibility to research the quality (e.g. type, etc.) of cannabis and quantity of the cannabinoid(s) on a scientific basis and to determine the origin of the cannabis. Some applications of the kit are e.g.:
CBD, CBN, THV and THC concentration analysis;
determination of the freshness of the cannabis samples;
possibility to make a menu of cannabis cultivars;
tracking the cannabinoid levels during the growth of the plants;
determination of the variation of the (neuro-) pharmacological, physiological and psychoactive cannabinoids in the users favorite marihuana sample or hashish specimen can be determined;
determination of the relative concentration of the different cannabinoids in the users sample and make the connection to its “overall” effect;
monitoring by the pharmacist of the percentage rate of change of the cannabinoids during storage, in terms of the amount of acids present and the presence of the degradation products (due to aging and oxidation) of THC (=CBN) and the forming of proposed/suspected inactive degradation products of CBD (=Canabielsoin) and CBC (=Cannabicyclol). The amount of acids is an indication of the freshness of the strain (variety). For example, CBN is a breakdown product of THC; CBN makes the strain heavy sleepy etc. On the other hand, special patients groups need only CBD varieties. This shows the advantage of the method and kit for a quality and quantity test (e.g. for determining the ‘strength’ of the strain);
for horticulturists and breeders the percentage rate of change of the cannabinoids in the profile and THC percentage by changing environmental parameters during the growth stage and the difference in the cannabinoid profiles of your offspring and compare the profiles with the parents can be determined. Varieties can be recognized, etc.;
customs may determine the origin (state, breeder, etc.) of confiscated cannabis since each cannabis type has its own fingerprint (see also e.g. U.S. Pat. No. 5,252,490) which depends amongst others upon breeding conditions and the type of cannabis.

The cannabinoid(s) detection kit will replace the need for very expensive and time consuming commercial available tests in commercial laboratories. Qualitative and quantitative control and knowledge is a necessity for anyone who is using marijuana on the base of prescription by physician or using marihuana for recreational use, were e.g. high THC levels (=a favorite and personal fingerprint) in the samples is required.

The kit and method provide information by testing the concentration of the main psychoactive compound THC in the marihuana samples used, together with information of the more important working behavior of the combinatorial effects of CBD and CBN on THC and many other cannabinoids. This kit quantitative measures the cannabinoids THC, CBD, CBN, THV and over 15 other cannabinoids in a sample of marihuana or hashish.

Hence, the method includes advantageously a total or almost total cannabinoid-extraction protocol and a developing protocol that separates substantially all cannabinoids from THC varieties and other cannabinoids present on a TLC plate, e.g. a small glass plate coated with silica gel G60. The method needs only a very small amount of sample (e.g. only about 10-100 mg), a very small amount of extraction solvent (e.g. only about 1 ml) and a very small amount of developing solvent (e.g. only about 2 ml) per sample. The kit enables everyone to avoid laborious, time consuming and expensive analytical research and determine the cannabinoid content with the THC test and Cannabis fingerprint kit.

In another embodiment, the method for the detection of a cannabinoid in cannabis comprises:
a) extracting a sample of cannabis with an extraction solvent to provide an extract;
b) providing at least part of the extract to a thin layer chromatography plate;
c) developing a chromatogram by performing thin layer chromatography, thereby using a developing solvent, wherein the developing solvent comprises one or more halogen substituted hydrocarbons selected from the group consisting of halogen substituted ethane, halogen substituted propane and halogen substituted butane, wherein the hydrocarbon comprises at least two halogen groups, and wherein the developing solvent optionally further comprise chloroform; and
d) determining from the developed chromatogram and reference data the presence and optionally the amount of the cannabinoid in the cannabis.

In a further embodiment, the halogen substituted hydrocarbon is selected from the group consisting of R₁R₂R₃C′—Cₙ(Rₓ,R_{y})₂ₙ—C″R₄R₅R₆, wherein R₁, R₂, R₃, R₄, R₅, R₆, Rₓ and R_{y} are independently selected from H, OH, F, Cl, Br and I, and wherein at least one of R₁, R₂ and R₃ and at least one of R₄, R₅ and R₆, independently of each other are selected from the group consisting of F, Cl, Br and I, and wherein n is 0, 1 or 2.

In a further embodiment, at least two of R₁, R₂ and R₃ and at least two of R₄, R₅ and R₆, independently of each other are selected from the group consisting of F, Cl, Br and I.

In yet further variants, at least R₁=R₄, or in a variant at least R₁=R₄ and R₂=R₅, or in another variant at least R₁=R₄, R₂=R₅ and R₃=R₆ (i.e. C′ and C″ are symmetrically substituted).

In yet another embodiment, R₁, R₂, R₃, R₄, R₅ and R₆, are selected from the group consisting of F and Cl. Embodiments are e.g. 1,2-dichloro ethane, 1,1,2,2-tetrachloro ethane, 1,2-dichloro-1,2-dichloro ethane; 1,3-dichloro propane, 1,1,3,3-tetrachloro propane, 1,3-dichloro-1,3-dichloro propane; 1,2,3-trichloro propane, 1,1,2,3,3-pentachloro propane, 1,1,2,2,3,3-hexachloro propane; 1,3-dichlor-2-fluoro propane, 1,3-dichloro-1,2,3-trifluoro propane, 1,2,3-trichloro-1,2,3-trifluoro propane; 1,4-dichloro butane, 1,1,3-tetrachloro butane, 1,4-dichloro-1,4-dichloro butane; etc.

In yet a further embodiment, also isomers are included of above mentioned halogen substituted hydrocarbons (like halogen substituted isobutene).

In another embodiment, the developing solvent comprising one or more of these hydrocarbon substituted hydrocarbons R₁R₂R₃C′—Cₙ(Rₓ,R_{y})₂ₙ—C″R₄R₅ R₆ further comprises chloroform. In an embodiment, the developing solvent comprises at least 25 vol. % of one or more hydrocarbon substituted hydrocarbons R₁R₂R₃C′—Cₙ(Rₓ,R_{y})₂ₙ—C′R₄R₅R₆. In an embodiment, the developing solvent comprises at least 25 vol. % chloroform and at least 25 vol. % of one or more hydrocarbon substituted hydrocarbons R₁R₂R₃C′—Cₙ(Rₓ,R_{y})₂ₙ—C″R₄R₅R₆. In yet a further embodiment, the developing essentially (≧90 vol %) consists of chloroform and one or more hydrocarbon substituted hydrocarbons R₁R₂R₃C′—Cₙ(Rₓ,R_{y})₂ₙ—C″R₄R₅R₆.

According to a further aspect of the invention there is provided a detection kit for the detection of a cannabinoid in cannabis comprising:
a) a container containing at least an extraction solvent for extracting a sample of cannabis;
b) a thin layer chromatography plate; and
c) a container containing at least a developing solvent for developing a thin layer chromatogram, wherein the developing solvent comprises one or more halogen substituted hydrocarbons selected from the group consisting of halogen substituted ethane, halogen substituted propane and halogen substituted butane, wherein the hydrocarbon comprises at least two halogen groups, and wherein the developing solvent optionally further comprises chloroform.

According to yet another aspect of the invention, there is provided a developing solvent for analyzing cannabis by thin layer chromatography comprising at least 25 vol. % chloroform and at least 25 vol. % one or more halogen substituted hydrocarbons selected from the group consisting of halogen substituted ethane, halogen substituted propane and halogen substituted butane, wherein the hydrocarbon comprises at least two halogen groups, wherein the vol. % are relative to the total volume of the developing solvent.

Herein, the term cannabis is known to the person skilled in the art and refers e.g. to cannabis samples or cannabis preparations of the marijuana (hemp) plant, Cannabis sativa. They include marijuana leaf, bhang, ganja, or hashish (derived from the resin of the flowering heads of the plant) and hashish oil. Cannabis may for instance be a green-gray mixture of dried, shredded flowers and/or leaves of the hemp plant. It is sometimes called pot, herb, weed, boom, grass, pot, dope, reefers, Mary Jane, gangster, chronic, etc. It can be smoked as a cigarette (called a joint or a nail) or in a pipe or bong.

### SPECIFIC EXAMPLES OF EMBODIMENTS

Embodiment 1

Referring to FIGS. 1-9, this embodiment describes in detail a way in which a fingerprint of the cannabis can be obtained.

Step 1: Weighing and Sampling

The cannabis (or hashish) are added to a cannabis sample container 1, in FIG. 1 an Eppendorf reaction tube, designed such that a predetermined amount can be added to the container 1. The container has a mark 3 for indicating a 100 mg (predetermined amount) cannabis sample 2.

In stead of using a container 1 designed such that a predetermined amount can be added to the container, a balance e.g. an analytical balance may be used.

Step 2: Adding Extraction Solvent

From an extraction solvent container 4 (with cover 4a) with another container 6 a predetermined amount of extraction solvent 5, in FIG. 2 a pipette, 1 ml (predetermined volume) extraction solvent 5 is added to the to the container 1 mentioned in step 1 (Eppendorf reaction tube). Of course, another container can be used, wherein both extraction solvent 5 and cannabis sample 2 are provided.

Step 3: Extraction

By closing this container 1 with cover 1a and shaking the container (by hand) for a few minutes, e.g. 2-4 minutes, the extraction solvent 5 and sample 2 are mixed in order to enable extraction, thereby providing an extraction solvent with extract 7. Extraction takes place in extraction container 8, which is in this embodiment the same container as the cannabis sample container 1.

Step 4: Filtration

Filtration is performed by arranging e.g. cotton 9 (like absorbent cotton/cotton wool) in the Eppendorf reaction (container 1) containing the extraction solvent with extract 7, in fluid contact with the liquid surface of the extraction solvent with extract 7. The cotton is placed “on top” of the greenish (due to chlorophyll) extraction solvent 7 and the cotton gets saturated with the extraction liquid (=filtration).

Step 5: Sampling Extraction Liquid

With a spotting container 10, in FIG. 3 a capillary tube, designed to sample a predetermined volume of the extraction liquid, a predetermined volume of the extraction liquid can be extracted to be spotted on the TLC plate 11, thereby providing one or more spot(s) 12 (see below). The capillary tube will take up the predetermined amount of extraction liquid, here 2 μl, by capillary action. An “end-to-end capillary” which is filled completely may be used.

Step 6: Spotting on TLC Plate

With the capillary tube 10, the TLC plate 11 is spotted with the liquid 7 contained in the container 8. Referring to the step above, 2 μl of sample. within the capillary tube with 2 μl of extraction liquid is spotted on the TLC plate, about 1 cm (h1) from the bottom of the plate 11 and about ½ cm (w1) or more from the side edges of the plate 11. The width w of the plate 11 is about 4 cm; the height h is about 10 cm.

Step 7: Developing the TLC Plate

A developing chamber or container 15 is provided and the TLC plate 11 is placed in the developing container 15. Before placing the TLC plate 11 in the developing chamber or container 15, developing solvent 14 is added, such that the bottom of TLC plate 11 (when placed) will be dipped in the fluid 14 for about 3-5 mm. The angle a between a normal to the surface and the plate 11 is between about 0 and 45°.

The developing chamber or container 15 is closed with cover 15a when both the developing solvent 14 (from a developing solvent container 13 with cover 13a) and TLC plate 11 are provided into the chamber or container 15. Then the chromatogram is allowed to develop for about 20 minutes. Development is finished by removing the plate 11 from the developing solvent 14 when the solvent front has reached the top of the plate or has at least traveled about 8 cm from the initial spot(s) 12.

Thereafter, the TLC plate 11 is removed from the developing chamber 15 and allowed to dry at ambient conditions.

Step 8: Preparation of the Visualizing Agent

Visualizing agent 18 (dye, colorizing agent or stain) from a container or vial 17 (cover 17a) is introduced into a nebulizer or sprayer 19 filled with (a predetermined amount) of a diluent, e.g. water) and are mixed. The visualizing agent 18 may also be present in the kit in a container 17 as ready spray solution, but preferably, a fresh dye/stain solution is made, preferably with diazo fast blue (Fluka no 44660). For example, a fresh 0.15-1/0% (w/v) diazo fast blue is prepared.

Step 9: Visualization Step

The agent 18 is sprayed on the plate 11. The bright colored cannabinoid spots 16 appear (nearly) instantaneously.

Step 10: Determination Step

From the Rf values (see above) or a reference TLC plate the presence of the cannabinoid(s) (and/or their corresponding acid(s)) is determined.

Embodiment 2

This embodiments describes a way in which the amount of THC, and in a similar way of the other cannabinoid(s), in the cannabis can be determined.

In general, the same protocol is followed as described above in embodiment 1, but with a number of modifications: Step 1*: Weighing and Sampling

See step 1 above. Further, the sample must be heated. There are a number of convenient ways to do this. One of them may be incorporated into step 1, others may be incorporated into step 6. Below, the incorporation of heating for step 1 is described.

The sample may be placed on a little piece of aluminum foil or on a small dish or in a crucible, etc. and is heated for about 4 minutes at 150° C.

Embodiment 3

Referring to FIG. 5, this embodiment describes alternative ways in which the amount of THC, and in a similar way of the other cannabinoid(s), in the cannabis can be determined.

Step 6*: Spotting on TLC Plate

See step 6 above. Further, the sample must be heated. Note that the heating of the sample will either be performed at step 1 (see embodiment 2) or after step 6 and before step 7, as described here.

After step 6, as described above, the TLC plate 11 can be heated. The same heating conditions as described above can be used, e.g. 4 minutes in an oven at 150° C. Alternatively, the TLC 11 plate may be carefully heated with a candle 21. Good results were obtained with a distance h4 of 3 cm and a heating time of 1½ minutes. Then, one can continue with developing the TLC plate 11, see above.

Embodiment 4

This embodiment describes an example of the detection kit according to the invention:

A box, preferably a cardboard box, containing:
one or more extraction solvent container(s) 4 containing the extraction solvent 5;
one or more TLC plate(s) 11;
one or more developing solvent container(s) 13 containing the developing solvent 14, with cover(s) 13a.
   Embodiment 5

This embodiment describes an example of the detection kit according to the invention:

A box, preferably a cardboard box, containing:
one or more extraction solvent container(s) 4 containing the extraction solvent 5;
one or more TLC plate(s) 11;
one or more developing solvent container(s) 13 containing the developing solvent 14, with cover(s) 13a.
one or more selected of the group consisting of a Rf data sheet, a RRf data sheet, a reference TLC sheet or copy thereof and reference data on a data carrier for loading on a computer, which, when loaded upon the computer provides information on Rf data (or RRf data or both) and/or a reference TLC sheet. Further, the data carrier may comprise a program, which when loaded on a computer, can compare a scanned developed TLC with the reference data and provide output information on the cannabinoid(s) present in the sample or the amount of the cannabinoid(s) present in the sample or both this qualitative and quantitative information.
   Embodiment 6

This embodiment describes an example of the detection kit according to the invention:

A box, preferably a cardboard box, containing:
one or more extraction solvent container(s) 4 containing the extraction solvent 5;
one or more TLC plate(s) 11;
one or more developing solvent container(s) 13 containing the developing solvent 14, with cover(s) 13a.
a container 17 with cover 17a, like a bag or a flask, containing a visualizing agent 18.

The kit may contain the visualizing agent 18, preferably diazo fast blue (Fluka no. 44660) as ready composition to be sprayed on the plate or as compound to be diluted. In a variant, the kit may contain a sprayer 19 filled with the visualizing agent 18, ready to be sprayed on a developed plate 11.

Embodiment 7

This embodiment describes an example of the detection kit according to the invention:

A box, preferably a cardboard box, containing:
one or more extraction solvent container(s) 4 containing the extraction solvent 5;
one or more TLC plate(s) 11;
one or more developing solvent container(s) 13 containing the developing solvent 14, with cover(s) 13a.
a UV lamp 20.
   Embodiment 8

This embodiment describes another example of the detection kit according to the invention:

A box, preferably a cardboard box, containing:
one or more container(s) 1, e.g. Eppendorf reaction tube(s), with a mark 3 for indicating a content of 100 mg and having a volume of about 1.5 ml;
one or more extraction solvent container(s) 4 containing the extraction solvent 5;
one or more pipette(s) 6, designed to contain a predetermined volume of extraction liquid;
one or more filter means 9 e.g. cotton or filter paper(s);
one or more extract sampling means or spotting container(s) 10 like syringe(s), pipette(s), or capillary tube(s), designed to contain a predetermined volume of the extraction liquid with extract 7;
one or more TLC plate(s) 11;
one or more developing solvent container(s) 13 containing the developing solvent 14, with cover(s) 13a;
optionally one or more selected of the group consisting of a lighter 21, a candle 21, a heater and a small furnace.
a TLC developing container 15;
a container containing a visualizing agent 17, like a bag or a flask;
a sprayer 19;
one or more selected of the group consisting of a Rf data sheet, a RRf data sheet, a reference TLC sheet or copy thereof and reference data on a data carrier for loading on a computer, which, when loaded upon the computer provides information on Rf data (or RRf data or both) and/or a reference TLC sheet. Further, the data carrier may comprise a program, which when loaded on a computer, can compare a scanned developed TLC with the reference data and provide output information on the cannabinoid(s) present in the sample or the amount of the cannabinoid(s) present in the sample or both this qualitative and quantitative information.
   Embodiment 9

This embodiment describes another example of the detection kit according to the invention:

A box, preferably a cardboard box, containing:
one or more container(s) 1, e.g. Eppendorf reaction tube(s), with a mark 3 for indicating a content of 100 mg and having a volume of about 1.5 ml;
one or more extraction solvent container(s) 4 containing the extraction solvent 5;
one or more pipette(s) 6, designed to contain a predetermined volume of extraction liquid;
one or more filter means 9 e.g. cotton or filter paper(s);
one or more extract sampling means or spotting container(s) 10 like syringe(s), pipette(s), or capillary tube(s), designed to contain a predetermined volume of the extraction liquid with extract 7;
one or more TLC plate(s) 11;
one or more developing solvent container(s) 13 containing the developing solvent 14, with cover(s) 13a;
optionally one or more selected of the group consisting of a lighter 21, a candle 21, a heater and a small furnace.
a TLC developing container 15;
a UV lamp;
one or more selected of the group consisting of a Rf data sheet, a RRf data sheet, a reference TLC sheet or copy thereof and reference data on a data carrier for loading on a computer, which, when loaded upon the computer provides information on Rf data (or RRf data or both) and/or a reference TLC sheet. Further, the data carrier may comprise a program, which when loaded on a computer, can compare a scanned developed TLC with the reference data and provide output information on the cannabinoid(s) present in the sample or the amount of the cannabinoid(s) present in the sample or both this qualitative and quantitative information.
   Embodiment 10

In the method of the invention and the detection kit of the invention, there is provided a developing solvent 14, which is also used as extraction solvent 5. To this end, the kit of the invention may comprise one or more container(s) 4/13. Developing solvent 14/extraction solvent 5 comprises at least 25 vol. % chloroform and at least 25 vol. % 1,2-dichloro ethane, relative to the total volume of the developing solvent 14/extraction solvent 5, the remainder one or more other organic solvents known to the person skilled in the art.

In a variant, developing solvent 14/extraction solvent 5 contains 25-75 vol. % chloroform and 75-25 vol. % 1,2-dichloro ethane.

In another variant, the following solvents were used as developing solvent 14:
1—Petroleum ether (40-60° C.) +1,5% Isopropyl alcohol (IPA)
2—Heptane—1% IPA (70:30)
3—Petroleum ether—ether (4:1)
4—Chloroform (ethanol free) 1,1-dichloro ethane
5—Xylene—1,4-dioxan (19:1)

In a variant, these developing solvents are combined with a mixture of chloroform and 1,2-dichloro ethane. Developing solvents 14 containing 25-75 vol. % chloroform and 75-25 vol. % 1,2-dichloro ethane (e.g. 60:40) give good results.

Embodiment 11

TLC plates are scanned with a commercial scanner or an image is made with a CCD camera. With data processing (software) and with e.g. Labview, a fully atomized qualitative and quantitative analysis protocol can be generated. To this end video densitometric methods are applied for counting pixels and with using calibration series of e.g. THC, CBD, CBN en CBG, these compounds and their concentrations can be detected. As reference plates for qualitative and quantitative plates, similar plates like plates 1 and 2 in FIG. 8 (qualitative analysis/fingerprint analysis), and plate 2 from FIG. 8 and the reference sheet from FIG. 9 can be used (for quantitative analysis; note that the plate displayed in FIG. 9 relates to THC; similar plates can be provided for other cannabinoids), respectively.

Embodiment 12

In this embodiment, 1,1,2,2-tetrachloro ethane can be used as developing solvent, either alone or in combination with e.g. chloroform. Further, the same method of detection and detection kit, as described in above embodiments can be used, with the exception that reference data are related to this developing solvent.

Embodiment 13

This embodiment describes an example of the detection kit according to the invention:

A box, preferably a cardboard box, containing:
one or more extraction solvent container(s) 4/developing solvent container(s) 13 containing the extraction solvent 5 (i.e. the extraction solvent is also used as developing solvent);
one or more TLC plate(s) 11;
   Embodiment 14

Below, some embodiments of (HP)TLC plates are given. Preferred embodiments are directed to those with silica 60 or silica G-60 as thin layer material. The (HP)TLC plate support material may comprise e.g. glass, aluminum, a plastic or a polyester. Examples are e.g. MERCK art.# 1.05719 (0.0001) plates of 5*10 cm (silica gel F254) and SIGMA art.# Z29,299-0 (silica gel 60, 5*10 cm) or analogous plates. In an embodiment, Macherey Nagel (MN) art # 7850956 ADAMANT plates (silica gel) may also be used.

The (HP) thin layer material preferably comprises silica, but may also comprise cellulose (e.g. with C₈ (RP-8) or C₁₈ (RP-18) side chains), as known to the person skilled in the art, aluminum oxide, kieselguhr or polyamide.

A non-limitative summary is given of HP(TLC) thin layer material:
Silica gel (e.g. Silica gel 60, Silica gel 60 W, Silica gel 60 F254, Silica gel 60 WF254s, Silica gel F254, Silica gel 60 F254 multiformat, Silica gel 60 silanized, Silica gel 60 F254 silanized, Silica gel 60 F254s caffeine impregnated;
Cellulose (e.g. Cellulose, Cellulose F, PEI Cellulose, PEI Cellulose F);
CHIR (also pure paper, without support material may be used);
PEI;
CN (e.g. CN, CN F254s);
DIOL (e.g. DIOL, DIOL F254s);
NH2 (e.g. NH2, NH2 F254s);
RP-2 (e.g. RP-2, RP-2 F254s);
RP-8 (e.g. RP-8, RP-8 F254s);
RP-18 (e.g. RP-18, RP-18 W, RP-18 F254s, RP-18 F254s (1 mm), RP-18 WF254s);
Aluminum oxide (Aluminum oxide 60, Aluminum oxide 60 F254, Aluminum oxide 60 F254 (neutral), Aluminum oxide 60 F254 (1.5 mm); Aluminum oxide 150, Aluminum oxide 150 F254, Aluminum oxide 150 F254 (neutral), Aluminum oxide 60 F254 (1.5 mm));
Kieselguhr (e.g. Kieselguhr, Kieselguhr F254);
Polyamide (polyamide derivatized silica) (e.g. Polyamide 11, Polyamide 11 F254)

Also Silica gel 150 analogues to the above 60 silica gels may be used.

Herein:

60, 150: Pore diameters in Angstroms
CHIR: Chiral plate with C18 modification; impregnation with copper salt and an optically active hydroxyproline derivative
CN: Cyano derivatized silica
DIOL: Glycerol derivatized silica
F Fluorescent indicator activated at 254 nm, emission at 523 nm
Fs: Contains acid stable fluorescent indicator, activated at 254 nm
NH2: Amino derivatized silica
PEI: Polyethyleneimine derivatized cellulose (store at 0-10° C.)
RP-2: dimethylsilyl derivatized silica (C1)
RP-8: derivatized silica (C8)
RP-18: derivatized silica (C18)
W: Water stable, able to accommodate mobile phase

The person skilled in the art may also select other plates with similar properties for application of the invention. Characteristic layer thicknesses of the (HP) TLC plate material (on the support material) are e.g. between about 0.15 and 0.25 mm. For example, silica gel G-60 TLC plates were used with a layer thickness of 0.25 mm.

The embodiments described above and as schematically depicted in the drawings are not on scale. The person skilled in the art will understand that most of the depicted containers may also be of other types, e.g. flasks, tins, bags, vials, etc. Further, only those parts of the kit relevant for the invention are depicted and described. The scope of protection of the invention is not limited to the embodiments given. The invention resides in each novel characteristic and each combination of characteristics. Reference numerals in the claims do not limit the scope of protection thereof. The use of the verb “comprise” and its declinations does not exclude the presence of elements other than those specified in the claims. The use of the indefinite article “a” or “an” preceding an element does not exclude the presence of a plurality of such elements.

1. A method for the detection of a cannabinoid in cannabis comprising:
a) extracting a sample of cannabis with an extraction solvent to provide an extract;
b) providing at least part of the extract to a thin layer chromatography plate;
c) developing a chromatogram by performing thin layer chromatography, thereby using a developing solvent, wherein the developing solvent comprises chloroform and 1,2-dichloro ethane; and
d) determining from the developed chromatogram and reference data the presence and optionally the amount of the cannabinoid in the cannabis.

2. The method according to claim 1, wherein for determining the presence of the cannabinoid or the amount of the cannabinoid or both a visualizing agent is sprayed over the developed chromatogram.

3. The method according to claim 1, wherein for determining the presence of the cannabinoid or the amount of the cannabinoid or both a UV source is used for irradiating and visualizing a spot of the cannabinoid on the developed chromatogram.

4. The method according to claim 1 wherein the cannabinoid is selected from the group consisting of cannabichromene (CBC), cannabidol (CBD), cannabidivarol (CBDV), cannabigerol (CBG), cannabinol (CBN), tetrahydrocannabinol (THC), tetrahydrocannabichromene (THCBC), tetrahydracannabidol (THCBD), tetrahydrocannabidivarol (THCBDV), tetrahydrocannabigerol (THCBG), and tetrahydrocannabivarin (THV), and their corresponding acids.

5. The method according to claim 1, wherein the extraction solvent comprises chloroform.

6. The method according to claim 5, wherein the extraction solvent comprises at least 50 vol. % chloroform, relative to the total volume of the extraction solvent.

7. The method according to claim 1, wherein the developing solvent comprises at least 25 vol. % chloroform and at least 25 vol. % 1,2-dichloro ethane, relative to the total volume of the developing solvent.

8. The method according to claim 1, wherein the developing solvent consists of 25-75 vol. % chloroform and 75-25 vol. % 1,2-dichloro ethane, relative to the total volume of the developing solvent.

9. The method according to claim 1, comprising:
weighing a part of the cannabis such that a sample is provided with a predetermined weight; and optionally heating the sample;
mixing extraction solvent and the sample;
extracting at least part of the total amount of the cannabinoids in the cannabis sample with the extraction solvent;
optionally filtering the extract;
sampling the optionally filtered extraction liquid with extract to provide a sample to be spotted on the TLC plate;
spotting on the TLC plate an amount of the extraction liquid with the extracted cannabinoid(s); and optionally heating one or more spots on the TLC plate;
developing the TLC plate;
visualizing the separated component(s) on the developed TLC plate;
determining from the developed chromatogram and the reference date the presence, the amount or both the presence and the amount of the cannabinoid in the cannabis sample.

10. The method according to claim 1, wherein the developing solvent is also used as extraction solvent.

11. A detection kit for the detection of a cannabinoid in cannabis comprising:
a) a container containing at least an extraction solvent for extracting a sample of cannabis;
b) a thin layer chromatography plate; and
c) a container containing at least a developing solvent for developing a thin layer chromatogram, wherein the developing solvent comprises chloroform and 1,2-dichloro ethane.

12. The kit according to claim 11, wherein the extraction solvent comprises chloroform.

13. The kit according to claim 12, wherein the extraction solvent comprises at least 50 vol. % chloroform, relative to the total volume of the extraction solvent.

14. The kit according to claim 11, wherein the developing solvent comprises at least 25 vol. % chloroform and at least 25 vol. % 1,2-dichloro ethane, relative to the total volume of the developing solvent.

15. The kit according to claim 11, wherein the developing solvent consists of 25-75 vol. % chloroform and 75-25 vol. % 1,2-dichloro ethane, relative to the total volume of the developing solvent.

16. The kit according to claim 11, wherein the developing solvent is also used as extraction solvent and wherein the container containing at least an extraction solvent for extracting a sample of cannabis comprises the container containing at least a developing solvent for developing a thin layer chromatogram.

17. The kit according to claim 11, further comprising a medium comprising reference data, wherein the reference data comprise one or more of a) information on the retardation factor (Rf, RRF or both) of the cannabinoid, b) the relation of spot area of the cannabinoid on the developed chromatogram and amount of the cannabinoid in the cannabis, c) the relation of spot intensity on the developed chromatogram and amount of the cannabinoid in the cannabis and d) the relation of spot area and spot intensity of the cannabinoid on the developed chromatogram and amount of the cannabinoid in the cannabis.

18. The kit according to claim 11, wherein the kit further comprises:
d1) a container or bag containing a visualizing agent for the cannabinoid on the developed chromatograrn.

19. The kit according to claim 11, wherein the kit further comprises
d2) a UV source.

20. A developing solvent for analyzing cannabis by thin layer chromatography comprising at least 25 vol. % chloroform and at least 25 vol. % 1,2-dichloro ethane, relative to the total volume of the developing solvent.

21. The developing solvent according to claim 20 containing 25-75 vol. % chloroform and 75-25 vol. % 1,2-dichloro ethane, relative to the total volume of the developing solvent.

22. A method for the detection of a cannabinoid in cannabis comprising:
a) extracting a sample of cannabis with an extraction solvent to provide an extract;
b) providing at least part of the extract to a thin layer chromatography plate;
c) developing a chromatogram by performing thin layer chromatography, thereby using a developing solvent, wherein the developing solvent comprises one or more halogen substituted hydrocarbons selected from the group consisting of halogen substituted ethane, halogen substituted propane and halogen substituted butane, wherein the hydrocarbon comprises at least two halogen groups, and wherein the developing solvent optionally further comprises chloroform; and
d) determining from the developed chromatogram and reference data the presence and optionally the amount of the cannabinoid in the cannabis.

23. A detection kit for the detection of a cannabinoid in cannabis comprising:
a) a container containing at least an extraction solvent for extracting a sample of cannabis;
b) a thin layer chromatography plate; and
c) a container containing at least a developing solvent for developing a thin layer chromatogram, wherein the developing solvent comprises one or more halogen substituted hydrocarbons selected from the group consisting of halogen substituted ethane, halogen substituted propane and halogen substituted butane, wherein the hydrocarbon comprises at least two halogen groups, and wherein the developing solvent optionally further comprises chloroform.

24. A developing solvent for analyzing cannabis by thin layer chromatography comprising at least 25 vol. % chloroform and at least 25 vol. % one or more halogen substituted hydrocarbons selected from the group consisting of halogen substituted ethane, halogen substituted propane and halogen substituted butane, wherein the hydrocarbon comprises at least two halogen groups, relative to the total volume of the developing solvent.
